(19) United States
(12) Patent Application Publication
(10) Pub. No.: US 2023/0334549 A1
(43) Pub. Date: Oct. 19, 2023

(54) METHOD FOR SELECTING A DENTAL PRODUCT

(71) Applicant: DENTAL MONITORING, Paris (FR)

(72) Inventors: Thomas PELLISSARD, MAISONS-ALFORT (FR); Philippe SALAH, PARIS (FR)

(21) Appl. No.: 18/025,024

(22) PCT Filed: Sep. 8, 2021
(86) PCT No.: PCT/EP2021/074709

§ 371 Date: Mar. 7, 2023

(30) Foreign Application Priority Data
Sep. 8, 2020 (FR) FR2009099

(51) Int. Cl. G06Q 30/0601; G06T 7/00; A61B 1/24

(52) U.S. Cl. CPC G06Q 30/0631; G06T 7/0012; A61B 1/24; G06T 2207/30036; G06T 2207/20021; G06T 2207/20084; G06T 2207/20081; A61B 5/0059

(57) ABSTRACT

A method for selecting a dental product. The method includes: a) acquiring an updated image representing a dental arch of a target consumer, and optionally, first additional information; b) analyzing the updated image by a processing computer, so as to determine a value for a dental attribute relating to the dental situation; c) selecting as a function of the value, and optionally of the first additional information and optionally of second additional information stored, prior to step a), in a database, —a relevant dental product in a database, and, preferably—a relevant dental care professional in a database; d) presenting the consumer with a response relative to the relevant dental product and, preferably, to the relevant dental professional, and/or—delivering the relevant dental product to the consumer, and/or—placing the consumer in contact with a dental care professional selected in step c).

### TECHNICAL FIELD

The present invention relates to a method for selecting a dental product.

### PRIOR ART

Many dental products, such as toothpastes, are commercially available. Generally, the consumer does not necessarily specifically know the effect of all the dental products, or the desired effects in their particular dental situation. Therefore, they often select a dental product depending on the brand or the price, without knowing whether this product is the most suitable for their dental situation.

Maintaining oral hygiene, in particular without dental or orthodontic treatment, therefore is not optimal.

Therefore, a requirement exists for a method allowing the consumer to select a dental product that is best suited to their dental situation.

An aim of the present invention is to at least partially address this requirement.

### SUMMARY OF THE INVENTION

The invention provides a method for selecting at least one dental product for a target consumer, said method comprising the following steps:
a) acquiring, at an acquisition instant and with an acquisition device:
   at least one image, called “updated image”, at least partially depicting a dental arch of the target consumer; and
   optionally, first additional information;
b) analyzing the updated image by a processing computer, so as to determine a value for at least one dental attribute relating to the dental situation of the target consumer at the acquisition instant;
c) selecting, by the processing computer, as a function of said value, and optionally of said first additional information and optionally of second additional information that is stored, prior to step a), in a database:
   at least one dental product, called “relevant dental product”, from a database of dental products; and, preferably
   at least one dental care professional, called “relevant dental care professional”, from a database of dental care professionals;
d) presenting the target consumer with a response relating to said at least one relevant dental product and/or to said at least one relevant dental care professional, preferably by sending a message on a screen of said acquisition device, preferably on a screen of a mobile telephone used in step a); and/or
   delivering said at least one relevant dental product to the target consumer; and/or
   placing the target consumer in contact, preferably via videoconferencing, with a dental care professional, preferably with a dental care professional selected in step c).

As will be seen in further detail throughout the remainder of the description, a method according to the invention allows simple and rapid selection of a dental product, and preferably of a dental care professional, adapted to the specific dental situation of the target consumer. For example, the target consumer can take photos or a video of their teeth in front of the toothpaste aisle and immediately receive a response showing them a toothpaste and, if necessary, a dentist, adapted to their dental situation. They can also, alternatively or in addition, receive a package containing this toothpaste.

A method according to the invention can further comprise one or more of the following optional feature(s):
said at least one relevant dental product is a “general public” dental product, with a general public dental product being a dental product that can be suitable for a large number of individuals without requiring particular adaptation for each individual; in particular, an orthodontic appliance is not a general public dental product, as it is defined, adapted, and manufactured for a particular situation for a particular individual, unlike a toothpaste, for example, and unlike an orthodontic appliance, for example, a general public dental product is freely accessible for sale and does not require professional dental, dentistry, and/or orthodontics training in order to be used or consumed;
said at least one relevant dental product comprises a consumable, such as a toothpaste or a lotion;
said at least one relevant dental product is manufactured industrially, on an automated production line;
said at least one relevant dental product is selected from among toothpastes, toothbrushes, dental floss, chewing gums, interdental brushes, whitening kits, gum care products (in particular for preventing inflammation or reducing sensitivity), products for preventing bad breath and oral lotions;
said at least one relevant dental product is selected from among lip restructuring products, wrinkle reduction products (for example, hyaluronic acid or Botox kits);
step a), preferably steps a) and d), preferably steps a) to d), are at least partially, preferably completely, carried out by means of a dedicated application loaded into the acquisition device,
the acquisition device preferably being:
   a mobile telephone; or
   a device comprising a mirror, a camera or an imaging device, an acquisition computer and, preferably, a detector arranged to detect the presence of a person in front of the mirror, with the mirror preferably being one-way and the camera preferably being concealed behind the mirror;
the method
   comprises, before step a), a step in which the dedicated application is downloaded into the mobile telephone, with the downloading being triggered by reading a code printed on a dental product and/or on a panel arranged in an aisle in which dental products are arranged; or
   comprises, in step a), the detection, by said detector, of the target consumer in front of the mirror and the acquisition of the updated image when the target consumer is detected in front of the mirror, preferably detected in a predetermined position;
in step a), the dedicated application analyzes the content of a preview image, preferably displayed on a screen of the acquisition device, and guides the positioning of the target consumer as a function of the result of said analysis;
the additional information, i.e., the first and/or second additional information, includes additional information:
   defining a constraint or a preference to be imposed for the selection in step c); and/or
   defining a constraint or a preference to be imposed for the presentation and/or the delivery and/or the placing in contact in step c); and/or
   defining a dental objective to be achieved by the target consumer; and/or
   relating to the target consumer;
the additional information defining a constraint specifies:
   a maximum price, with the price of a dental product having to be below said maximum price so that said dental product can be selected in step c);
   when the dental product comprises a consumable, such as a toothpaste or a lotion, a range for the amount of the consumable, within which range the amount of consumable of a dental product must be included so that said dental product can be selected in step c);
   a maximum period for delivering or providing a dental product so that said dental product can be selected in step c), for example, to indicate that the target consumer wants the selected dental products to be immediately accessible, for example, available in the supermarket where they are located;
   a list of one or more ingredient(s) that a dental product must not include or must include so that said dental product can be selected in step c);
   a list of one or more technical feature(s) that a dental product must not include or must include so that said dental product can be selected in step c), for example, to exclude or be limited to electric toothbrushes;
   a list of manufacturing countries where a dental product must be manufactured or must not be manufactured so that said dental product can be selected in step c);
   a list of manufacturers by which a dental product must be manufactured or must not be manufactured so that said dental product can be selected in step c);
   a list of brands that must include the brand of a dental product so that said dental product can be selected in step c);
   a maximum price, with the price for a service by a dental care professional having to be below said maximum price so that said dental care professional can be selected in step c);
   a maximum period for making an appointment with the dental care professional so that said dental care professional can be selected in step c);
   a list of one or more item(s) of equipment that a dental care professional must have available so that said dental care professional can be selected in step c);
   a list of one or more diploma(s) or qualification(s) that a dental care professional must hold so that said dental care professional can be selected in step c);
   a list of language(s) in which a dental care professional must be proficient so that said dental care professional can be selected in step c);
   criteria for presenting the response in step d), for example, for classifying the selected relevant dental products in increasing or decreasing price order, or by brands, or as a function of their level of relevance to the dental situation or of the additional information;
   criteria for delivering the one or more relevant dental product(s) in step d), for example, the delivery mode, the delivery date, the payment terms;
   criteria for contacting the relevant dental care professional in step d), for example, the communication mode, the contact date, or the payment terms; and/or the dental objective is selected from among the following objectives: teeth whitening, reducing tartar, and improving breath; and/or
the additional information relating to the target consumer specifies:
   their age; and/or
   their gender, and/or
   the one or more dental care professional(s) they have already consulted or they consult regularly; and/or
   whether or not they are wearing an orthodontic appliance, in which case they can be asked, in step a), to specify the nature of this orthodontic appliance or to remove this orthodontic appliance; and/or
   if they are predisposed to certain pathologies, for example, if they have fragile gums or if they are allergic to certain ingredients; and/or
   if they have followed or if they follow or if they are contemplating following a particular orthodontic treatment, in which case they can be asked to specify the nature of this orthodontic treatment and/or its duration and/or when they are undergoing this orthodontic treatment; and/or
   if they are currently undergoing dental treatment in order to achieve a dental objective and/or, if they have already previously used the method within the context of said dental treatment, details concerning said dental treatment; and/or
   whether or not they are content with their smile, their breath or their current dental situation;
   if they experience pain, preferably by indicating a pain intensity, for example, on a pain scale, and/or preferably by indicating the location of the pain, preferably by indicating one or more dental organ(s);
   if one or more teeth of the target consumer is/are sensitive to contact with heat and/or cold, preferably by indicating the one or more teeth, and/or preferably by indicating a sensitivity intensity, for example, on an intensity scale;
in step a), the acquired first additional information is stored in said database, and/or wherein the second additional information is provided by one or more additional information source(s) relating to the target consumer and differing from the acquisition device (which provides the first additional information);
in step b), the dental attribute is:
   an appearance parameter for a gum, the tongue, one or two lips, a perioral area, a tooth or a group of teeth depicted on the updated image, in particular a translucency index or a color parameter, in particular relating to the white color,
   a parameter relating to the state of a gum, the tongue, one or both lips, a perioral area, a tooth or a group of teeth or the mouth depicted on the updated image, for example, relating to an inflammation, to the presence of tartar, to a breakage, to an abrasion, to cracking, to a lack of saliva, to a pathology, for example, a carie, or to the presence of a dental appliance, for example, an orthodontic appliance;
   a parameter relating to the shape of a gum, the tongue, one or both lips, a perioral area, a tooth or a group of teeth depicted on the updated image, for example, a mesial and distal deflection index of the incisal edge;
in step b), the following steps are carried out:
   segmenting, preferably by means of a “segmentation” neural network, so as to isolate the depiction of at least one dental organ on the updated image;
   determining the value of the dental attribute by submitting said depiction as input for a “valorization” neural network that is trained to provide, as output, a value of said dental attribute in response to the presentation, as input, of a depiction of one of said dental organs;
in step c), a set of rules is applied to the value determined in step b), and optionally to the additional information, with said set of rules comprising at least one rule selected from among:
   filtering rules suitable for retaining dental products of the database of dental products and/or for retaining dental care professionals of the database of dental care professionals, as a function of the value determined in step b) and optionally of the additional information; and
   weighting rules suitable for giving weights to the dental products of the database of dental products and/or to the dental care professionals of the database of dental care professionals, as a function of the value determined in step b) and optionally of the additional information;
the rules involve,
   said value determined in step b); and/or
   an item of said additional information, in particular a constraint and/or a dental objective and/or a preference and/or a requirement of the target consumer and/or a specific feature of the target consumer and/or a feature of an event prior to step a), for example, a feature relating to the reaction of the target consumer following a previous implementation of steps a) to d) and/or relating to the result of a previous implementation of steps a) to d) and/or relating to a temporal evolution of said value determined in step c); and/or
   a plurality of dental products and/or a plurality of dental care professionals, with a rule particularly being able to involve one or more dental product(s) and/or a plurality of dental care professionals that have met the prior application of other rules; and/or
   independent data of the target consumer,
the database in which the first additional information and optionally the second additional information is stored includes a record containing an identifier of the target consumer and, in step a), said identifier is determined by facial recognition;
in step c), a selection neural network is implemented in order to provide, as output, the one or more relevant dental product(s) and/or relevant dental care professional(s) as a function of the value determined in step b) and, optionally, of the additional information, introduced at the input of said selection neural network;
in step d), preferably by videoconferencing, preferably instantaneously, the target consumer and a dental care professional are placed in contact as a function of the value determined in step c), for example, in case of the detection of a carie, or only if the value determined in step c) is greater than or less than a threshold value;
the processing computer is programmed for, in step b):
   measuring the value of the dental attribute on the updated image; and/or
   segmenting the updated image and measuring the value of the dental attribute on a segment of the updated image;
the value of the dental attribute qualifies or quantifies the general dental situation of the target consumer and/or a state of an orthodontic appliance depicted on the updated image and/or a state of a tooth and/or a gum of the target consumer depicted on the updated image and/or the oral hygiene of the target consumer;
the additional information includes data relating to the target consumer that cannot be determined by the analysis of the updated image;
before step c), the processing computer assesses the reaction of the target consumer following a previous step d), then, in step c), selects, as a function of the assessment of said reaction, the one or more relevant dental product(s) and/or the one or more relevant dental care professional(s);
before step d), a company with a centralized computer programmed to execute at least steps c) and d) from a plurality of requests each containing a value of said at least one dental attribute, preferably sent by a plurality of target consumers, preferably by means of their mobile telephones, selects and/or sets the rules;
in one embodiment, the processing computer is integrated into the acquisition device, in particular the mobile telephone;
in one embodiment, the processing computer is remote from the acquisition device, in particular from the mobile telephone, and, in particular, can be in the assessment center with which the acquisition device communicates, with the assessment center preferably computationally processing requests originating from more than 100, more than 500, more than 5,000, more than 10,000, preferably more than 100,000 target consumers.

The invention also relates to a system comprising an acquisition device, preferably a mobile telephone, and a processing computer configured to implement steps a) and d), and steps b), c), respectively.

The invention also relates to:
a computer program, and in particular a dedicated application for a mobile telephone, comprising program code instructions for executing steps a), and/or b), and/or c) and/or d), when said program is executed by a computer,
a computer medium, on which such a program is stored, for example, a computer memory or a CD-ROM; and
a computer, in particular a mobile telephone, in which such a program is loaded.

### Definitions

“Target consumer” is understood to mean any person for which a method according to the invention is implemented, whether or not this person is ill.

“Dental practice” includes any practice in which a dental care professional, in particular a dentist, a hygienist or a professional specialized in orthodontics or periodontics works, or any shop in which a consumer can, in particular, have their teeth whitened.

A “dental situation” defines a set of features relating to an arch of a person at an instant, for example, the position of the teeth, their shape, their color, the position of an orthodontic appliance, the presence of inflammations, etc., at this instant.

A “dental organ” is an element inside the mouth, and particularly a tooth or a gum.

“Mobile telephone” is understood to mean any portable device that allows photos or videos to be taken and that can communicate via a mobile telephone network.

“Image” is understood to mean a two-dimensional digital depiction, such as a photograph or an image extracted from a video. An image is formed by pixels.

A “preview image” is an image captured by a camera or an imaging device that is not acquired, i.e., not stored. Triggering the acquisition causes the preview image to be stored. The preview image may or may not be displayed, for example, on the screen of a mobile telephone.

“Segmentation” of an image is a conventional operation by which areas of the image meeting a criterion or resulting from (“segments”) processing are defined. For example, the photo of an arch can be segmented in order to define the areas of this image that depict teeth, or “teeth areas”. The segments are independent entities in that they can be selected individually, for example.

A “tooth area” of an image is part of said image that substantially exclusively depicts a tooth, i.e., that follows the contour of this tooth on this image. In other words, the depiction of said tooth on the image is substantially 100% of the tooth area.

An “angle” is an orientation of the optical axis of a camera or an imaging device, for example, of a mobile telephone, relative to the target consumer when acquiring a photo. An object or a person, for example, a dental product or a dental care professional, have a physical nature. When they are referenced in a database or are selected or weighted, this involves computer records for this object or this person that are referenced or selected or weighted. For the sake of clarity, however, these records are designated by these objects or persons. For example, the “dental products” database contains records relating to dental products. If a “dental product is weighted”, a weight is given to its record. If the selection neural network “provides”, as output, the one or more relevant dental product(s) and/or dental care professional(s), it provides records relating to these dental products and dental care professionals. The context makes it possible to determine whether the designation of the object or of the person refers to this object or person or refers to a computer record relating to this object or person.

A method according to the invention is implemented by a computer, preferably exclusively implemented by a computer.

“Computer” designates a computer processing unit, which includes a set of a plurality of machines with computer processing capabilities. This unit particularly can be a mobile telephone or can be a PC-type computer or a tablet computer. If the unit comprises a plurality of machines, these machines include communication means for exchanging with each other. The computer can be a server remote from the target consumer, for example, it can be the “cloud” or can be a server of an “assessment center”.

Conventionally, a computer particularly comprises a processor, a memory, a human-machine interface, conventionally comprising a screen, a module for communicating via the internet, via Wi-Fi, via Bluetooth® or via the telephone network. Software configured to implement steps b), c) and d) is preferably loaded into the memory of the processing computer. A computer also can be connected to a printer.

A “neural network” or an “artificial neural network” is a set of algorithms well known to a person skilled in the art. A “learning database” is a database of computer records suitable for training a neural network.

In order to be operational, a neural network is conventionally trained by a learning process, called “deep learning”, from a learning database. The learning database conventionally contains information relating to the two types of object that the neural network must learn to match.

The training can occur from a learning database made up of records each comprising a first object of a first type and a corresponding second object, of a second type. The first objects are presented as input for the neural network and the second objects are output from the neural network, thereby enabling the neural network to learn to provide, from any object of the first type, a corresponding object of the second type.

For example, the neural network can be provided with a set of records each containing an image (first object) and a relevant dental product selected for this image (second object), so as to configure the neural network so that when it is presented with an updated image as input, it provides a relevant dental product as output.

For example, each record of the learning database can include an image of a dental arch and a description identifying, in this image, the depictions of the teeth, or “tooth areas”, and the corresponding tooth numbers. After being trained, the neural network can thus identify, on an image presented thereto, the depictions of the teeth and the corresponding tooth numbers.

The second object can be an image, and in particular an image that is identical to the first object, but it is “labeled”, i.e., in which information, and in particular a value for a dental attribute, has been added, for example, in graphical form.

The training of a neural network is suitable for the intended aim and does not pose any particular difficulty to a person skilled in the art.

“Comprise”, “include” and “present” must be understood in a broad and non-limiting manner, unless otherwise indicated. Similarly, the selection of a dental product or of a dental care professional does not exclude the selection of several dental products and/or of several dental care professionals. The acquisition of an updated image does not exclude the acquisition of a plurality of updated images.

### BRIEF DESCRIPTION OF THE FIGURES

Further features and advantages of the invention will become more clearly apparent upon reading the following detailed description and with reference to the appended drawings, in which:

FIG. 1 illustrates the implementation of a method according to the invention; and

FIG. 2 illustrates the use of a dental retractor.

### DETAILED DESCRIPTION

An aim of a method according to the invention is the selection of one or more dental product(s). The dental products are preferably selected from among toothpastes, toothbrushes, dental floss, chewing gums, interdental brushes, whitening kits, gum care products (particularly for preventing inflammation or for reducing sensitivity), products for preventing bad breath and oral lotions.

The dental products can be selected from among lip restructuring or wrinkle reduction products.

The method comprises the aforementioned steps. It is described in detail hereafter in the particular, non-limiting case whereby a mobile telephone is used for step a). Except in the event of technical incompatibility, the features described for this particular case are applicable to other acquisition devices.

Prior to step a), a dedicated application is loaded into the mobile telephone of the target consumer.

The target consumer can receive an advertisement prompting them to download the dedicated application, for example, on their telephone, for example, on social networks. They can also download the dedicated application after they have been advised to do so by someone.

Preferably, the dedicated application is downloaded over the Internet. In particular, it can be downloaded from the Apple store or from the Google store.

In a preferred embodiment, downloading is triggered by reading a code, preferably a QR code, using the mobile telephone. The code particularly can be printed on the dental product packaging and/or on a panel arranged in the aisle where the dental products are arranged.

Preferably, if the dedicated application has already been loaded into the mobile telephone, a message, for example, an SMS, is sent to the target consumer to advise them to execute the method of the invention. Sending this message is particularly useful if, during a previous implementation of the method, the target consumer has specified that they wish to achieve a dental objective. A coaching program providing dates for executing the method then can be established in order to achieve this dental objective. Reminders are sent to the target consumer accordingly.

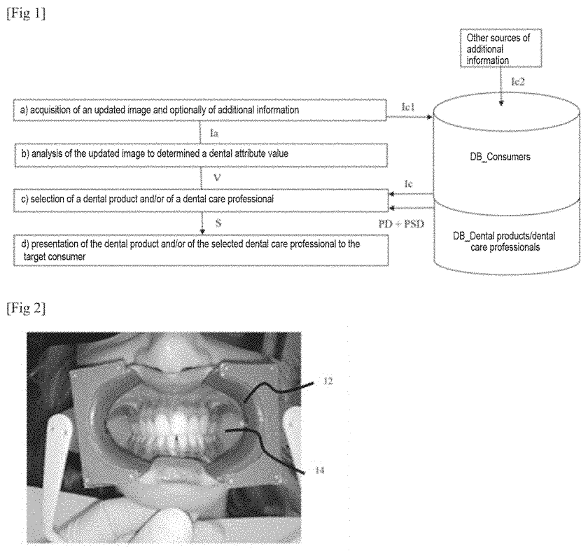

In step a), after having launched the dedicated application, an operator, preferably the target consumer or someone close to the target consumer, acquires, by means of the mobile telephone, at least one updated image Ia (see FIG. 1) depicting at least part of one of the dental arches of the target consumer. Step a) is preferably carried out by the target consumer. Step a) advantageously can be carried out remotely from any dental practice, for example, at the home of the target consumer or in a store or a pharmacy in which dental products are offered for sale or in which teeth whitening can be carried out.

Preferably, the updated image is a photograph, preferably with realistic colors. It depicts the mouth of the target consumer substantially as seen by an operator placed in the same location as the mobile telephone, and in particular with the same colors. In one embodiment, the updated image is extracted from a video.

The updated image is preferably an extra-oral image, preferably taken at a distance ranging between 5 cm and 40 cm from the mouth of the target consumer.

In one embodiment, the target consumer separates their lips to reveal their teeth. They can simply smile, or pull their lips with their fingers, or push them back with a tool, for example, with a spoon, or use a conventional dental retractor, as shown in FIG. 2. A dental retractor is a well-known device comprising channels 12 extending around an opening 14. The target consumer places their lips in the channels, which allows them to be separated and improves the observation of the dental arches through the opening.

In a preferred embodiment, no dental retractor is used in step a). The target consumer can advantageously implement the method at any time, provided they have their mobile telephone.

In one embodiment, the dedicated application guides the operator when they position the mobile telephone. To this end, it analyses, preferably by means of a neural network, the content of the preview image displayed on the screen of the mobile telephone in order to determine whether the angle of the mobile telephone and/or the distance between the mobile telephone and the mouth of the target consumer allows an updated image to be acquired that is well suited for the analysis to be carried out in step b). If this is not the case, it notifies the operator and, preferably, provides them with instructions for modifying said angle and/or said distance.

Preferably, the dedicated application activates the front camera, i.e., the camera that is on the same side of the mobile telephone as the screen. The target consumer can then observe the preview image before acquiring the updated image themselves, and can therefore better position the mobile telephone relative to the teeth they want to photograph.

In one embodiment, the target consumer uses the rear camera with the flash.

In step a), the mobile telephone can also acquire additional information. The additional information is preferably information that cannot be deduced from the analysis of the updated image, or that can only be deduced therefrom with difficulty. The additional information acquired in step a) is referred to as “first additional information” (reference “Ic1” in FIG. 1).

The first additional information can be sent directly to the processing computer that carries out step 2) and/or can be recorded, with an identifier of the target consumer or of the mobile telephone, in a “DB_Consumers” database that the processing computer has access to in order to execute step c).

An identifier of the target consumer can be entered by the target consumer. In one embodiment, a facial recognition algorithm identifies the target consumer without the target consumer having to interact with their mobile telephone.

Consulting the “DB_Consumers” database advantageously avoids having to ask the target consumer questions, during cycles of subsequent steps a) to d), for which the answer is already known.

The “DB_Consumers” database can be remote from the mobile telephone, for example, in the assessment center with which the mobile telephone communicates.

The “DB_Consumers” database preferably is also fed from other additional information sources, for example, from a database of the store in which the target consumer is located and which lists their prior purchases. The additional information originating from these other sources is referred to as “second additional information” (reference “Ic2” in FIG. 1).

The first and second additional information are collectively referred to as additional information Ic.

The additional information can specify particular constraints that the target consumer wants to impose for the selection in step c).

A constraint can be the exclusion of certain dental products or certain dental care professionals, for example, by specifying:
for the dental products that are likely to be selected,
   a maximum price;
   when the dental product comprises a consumable, such as a toothpaste or a lotion, a range for the amount of consumable;
   a maximum delivery period, for example, to indicate that the target consumer wants the selected dental products to be immediately accessible, for example, available in the supermarket where they are located;
   a list of one or more ingredient(s) that the dental products that are likely to be selected must include or must not include;
   a list of one or more feature(s) that the dental products that are likely to be selected must include or must not include, for example, to exclude or be limited to electric toothbrushes;
   a list of manufacturing countries to be excluded or favored;
   a list of manufacturers from manufacturing countries to be excluded or favored;
for dental care professionals that are likely to be selected,
   a maximum price for the consultation;
   a maximum distance from the position of an object or a person, for example, relative to the position of the target consumer at the acquisition instant of step a);
   availability, for example, for an appointment to be possible within a period that is less than a maximum period;
   possession of a particular item of equipment;
   a list of one or more dental care professional(s) to be excluded or from which the selection of step c) must be made.

The additional information can also specify a frequency at which a dental product can be proposed, or a subscription formula for a service for offering dental products (for example, 3 months, 6 months, or 1 year).

The additional information can also specify one or more “dental” objectives, beyond the single requirement identified from the updated image. For example, the dental objective can be “whitening the teeth” or “reducing plaque”, or “reducing tartar”, or “reducing a soft tissue inflammation”, or “improving breath”, or “correcting/improving the alignment of the teeth”, or “improving the general appearance of the smile”, or “reducing snoring and/or sleep apnea” or “improving chewing”, or “improving the shape of the lips”, or “improving the appearance of the perioral area”.

The additional information can specify preferences of the target consumer, in particular for classifying the dental products and/or the dental care professionals. For example, the target consumer can specify that they prefer an electric toothbrush to a manual toothbrush, or that they prefer one dental care professional from another. Unlike a constraint, a preference is not imperative. A dental product or a dental care professional that are not preferred can be selected.

Finally, the additional information can contain features of the target consumer, for example:
their age;
their gender;
whether or not they are wearing an orthodontic appliance, in which case they may be asked to specify the nature of this orthodontic appliance;
if they are predisposed to certain pathologies, for example, if they have fragile gums;
if they have followed or if they follow or if they are contemplating following a particular orthodontic treatment, in which case they may be asked to specify the nature of this orthodontic treatment and/or its duration and/or when they are undergoing this orthodontic treatment; and/or
if they are currently undergoing dental treatment in order to achieve a dental objective and/or, if they have already previously used the method within the context of said dental treatment, details concerning said dental treatment;
if they experience pain, in particular pain related to heat or cold, and/or a measure of the perceived pain, and/or a location of the perceived pain.

The additional information is preferably at least partially entered into the mobile telephone, preferably by presenting the target consumer with a list of options or questions, for example, in the form of check boxes.

In step b), a processing computer analyzes the updated image and the optional additional information that is acquired by the mobile telephone or, more generally, that is accessible in the DB_Consumers database.

Preferably, the processing computer is a computer of an assessment center, remote from the mobile telephone, that centralizes the processing of requests originating from a plurality of target consumers. Preferably, it receives requests from more than 1,000, more than 10,000, preferably more than 50,000, more preferably more than 100,000 target consumers.

In this latter case, a request is prepared by the dedicated application loaded into the mobile telephone. This request comprises the updated image acquired in step a) and optionally at least some of the first additional information. The request is transmitted to the processing computer by the mobile telephone. All the known communication means can be used to this end. In particular, the transmission can be carried out by a mobile telephone network with which the mobile telephone is subscribed. Conventionally, the request also contains information that allows the origin of the request to be identified, for example, an identifier, “ID_consumer”, of the target consumer.

In one embodiment, the processing computer is the mobile telephone, which means that a request does not need to be sent.

The processing computer analyzes the updated image in order to assess the dental situation of the target consumer. The value of an attribute relating to the dental situation of the target consumer, or “dental attribute”, allows this assessment to be quantified. Preferably, the processing computer determines the value of a plurality of dental attributes in order to improve the quality of the assessment of the dental situation.

The dental attribute particularly can be selected from the following list:
an appearance parameter for the tongue, one or two lips, a perioral area, a gum, a tooth or a group of teeth depicted on the updated image, in particular a translucency index or a color or texture or uniformity parameter (presence of stains);
a parameter relating to the state of the tongue, of one or both lips, of a perioral area, of a gum, of a tooth or of a group of teeth depicted on the updated image, for example, relating to an inflammation, a recession, the presence of tartar, a breakage, an abrasion, cracking, a pathology, for example, a carie, or to the presence of a dental appliance, for example, an orthodontic appliance;
a parameter relating to the shape of a gum, the tongue, the lip(s), a perioral area, a tooth or a group of teeth depicted on the updated image, for example, a mesial and distal deflection index of the incisal edge.

The dental attribute can be absolute, for example, it can relate to the whiteness of one or more teeth. Alternatively, the dental attribute can be relative, for example, it can relate to a color difference between two teeth or a gap between two adjacent teeth or an average gap between the teeth depicted on the updated image.

Determining the value of the dental attribute can also require a comparison reference that cannot be derived from the updated image. For example, the dental attribute can relate to a previous situation of the target consumer. The comparison reference can be, for example, the value of the dental attribute assessed during a previous cycle of step a) to d). For example, the dental attribute can be an assessment of the evolution of the color or of the position of one or more teeth since this previous cycle.

The dental attribute can relate to a standard dental situation, for example, determined by statistically processing previously measured data. For example, the dental attribute can be an assessment of a difference in shape with a standard model, for example, a typodont. It can measure, for example, a difference in tooth width, in particular of mesio-palatal width, thickness, or crown height with a standard model.

The dental attribute can be general. For example, the value of the dental attribute can be:
a “generally acceptable dental situation”, or a “generally unacceptable dental situation”; or
an “intact orthodontic appliance” or a “damaged orthodontic appliance”.

The dental attribute can be Boolean, i.e., only assume two values, for example, “acceptable color”, and “unacceptable color”. The values of a dental attribute also can be quantitative.

For example, a value of a dental attribute can define the extent to which a tooth has moved, has been abraded, or has changed appearance compared to a prior dental situation.

When the dental attribute relates to a particular dental organ, for example, a particular tooth, the analysis obviously must also determine this particular dental organ.

The analysis of the updated image can be carried out manually, preferably by a dental care professional. Preferably, it is carried out by a computer analysis. A person skilled in the art knows the applicable methods for analyzing images.

For example, the methods implementing the following operations can be cited:
comparing histograms (CalcBackProjectPatch, OTSU function);
measuring the difficulty of converting a normalized gray scale image into another image (EMD function);
comparing templates, which templates can be contours extracted from images (match template function);
computing the moment of a contour via the Green formula;
computing the invariant moment of a contour via the Hu formula;
comparing texture via descriptors (HOG, LBP, Haar);
comparing key points extracted from the image (SIFT/SURF/FREAK/ORB/ASIFT/AGAST function, etc.);
comparing Fourier transforms (DFT, FFT).

In particular, the analysis of step b) preferably comprises prior processing involving segmenting the updated image in order to determine the elements (or “segments”) that are depicted, for example, the tooth areas, the gum areas, or the orthodontic appliance areas.

The segmentation methods are well known. In particular, the use of a neural network, or “segmentation neural network”, is preferred.

For example, dedicated networks in the segmentation are:
Deep Mask (Learning To Refine Object Segments);
Mask-RCNN;
MaskLab (Instance Segmentation by Refining Object Detection with Semantic and Direction Features);
Fully Convolutional Instance-aware Semantic Segmentation.

Preferably, the segmentation neural network is the Mask R-CNN network. Such a neural network is described, in particular, in “Mask R-CNN. CoRR”, abs/1703.06870, 2017, Kaiming He, Georgia Gkioxari, Piotr Doll'ar, and Ross B. Girshick.

The learning database for training the segmentation neural network conventionally can be formed manually, by tracing and identifying all the depictions of the teeth depicted on historical photos. Each record of the learning database then includes a historical photo and a description of this photo identifying each of the areas of the historical photo (or “mask”) covering the depiction of a tooth. For training, each historical photo is provided at the input of the neural network, with its description being provided at the output of the neural network. The neural network thus learns to identify the tooth areas on a photo that it is presented with as input.

The segmentation results in a segmented image and a value of the dental attribute then can be measured on a segment. For example, after identifying the tooth area of tooth No. 3, i.e., the surface of the updated image that depicts this tooth, the processing computer can measure the color (dental attribute) of this tooth, and determine that it is “normal” (value of the dental attribute).

Determining the dental attribute preferably uses artificial intelligence algorithms, and in particular at least one neural network trained to this end. These algorithms are well known. The neural network, called “valorization neural network”, particularly can be selected from among:
networks specialized in the classification of images, called “CNN” (“Convolutional Neural Network”), for example:
   AlexNet (2012);
   ZF Net (2013);
   VGG Net (2014);
   GoogleNet (2015);
   Microsoft ResNet (2015);
   Caffe: BAIR Reference CaffeNet, BAIR AlexNet;
   Torch:
   VGG_CNN_S,VGG_CNN_M,VGG_CNN_M_2048,VGG_CNN_M_1024,VG G_CNN_M_128,VGG_CNN_F,VGG ILSVRC-2014 16-layer,VGG ILSVRC-2014 19-layer, Network-in-Network (Imagenet & CIFAR-10);
   Google: Inception (V3, V4).
the networks specialized in the location and detection of objects in an image, the Object Detection Network, for example:
   R-CNN (2013);
   SSD (Single Shot MultiBox Detector: Object Detection Network), Faster R-CNN (Faster Region-based Convolutional Network method: Object Detection network);
   Faster R-CNN (2015);
   SSD (2015);
   OverFeat (Sermanet et al.), 2013;
   GoogleNet (Szegedy et al.), 2015;
   VGGNet (Simonyan and Zisserman), 2014;
   R-CNN (Girshick et al.), 2014;
   Fast R-CNN (Girshick et al.), 2015;
   ResNet (He et al.), 2016;
   Faster R-CNN (Ren et al.), 2016;
   FPN (Lin et al.), 2016;
   YOLO (Redmon et al.), 2016;
   SSD (Liu et al.), 2016;
   ResNet v2 (He et al.), 2016;
   R-FCN (Dai et al.), 2016;
   ResNeXt (Lin et al.), 2017;
   DenseNet (Huang et al.), 2017;
   DPN (Chen et al.), 2017;
   YOLO9000 (Redmon and Farhadi), 2017;
   Hourglass (Newell et al.), 2016;
   MobileNet (Howard et al.), 2017;
   DCN (Dai et al.), 2017;
   RetinaNet (Lin et al.), 2017;
   Mask R-CNN (He et al.), 2017;
   RefineDet (Zhang et al.), 2018;
   Cascade RCNN (Cai et al.), 2018;
   NASNet (Zoph et al.), 2019;
   CornerNet (Law and Deng), 2018;
   FSAF (Zhu et al.), 2019;
   SENet (Hu et al.), 2018;
   ExtremeNet (Zhou et al.), 2019;
   NAS-FPN (Ghiasi et al.), 2019;
   Detnas (Chen et al.), 2019;
   FCOS (Tian et al.), 2019;
   CenterNet (Duan et al.), 2019;
   EfficientNet (Tan and Le), 2019;
   AlexNet (Krizhevsky et al.), 2012.

The above list is not limiting.

Preferably, the valorization neural network is the VGG Net network.

Preferably, the valorization neural network is trained using a learning database typically comprising more than 10,000, preferably more than 50,000, preferably more than 100,000 records, with each record containing a historical image, preferably a photo, of a “historical” dental arch, and a “historical” description providing values for one or more dental attribute(s).

Each historical image is presented at the input of the neural network, while the historical description is presented at the output of the neural network. The neural network thus learns to determine a description for a photo that it has been presented with as input.

In one embodiment, the analysis only uses the information provided by the updated image. The Applicant has developed a method implementing an artificial intelligence algorithm, preferably a neural network, and allowing, from a simple updated image of the arch of the target consumer, for example, a photograph taken by the target consumer with their mobile telephone, the dental situation of the consumer to be assessed. This method is described in EP 18184477.

A plurality of neural networks can be trained in order to each determine the value of a respective dental attribute. For example, if the dental objective is to select at least one product for improving breath, neural networks can be trained in order to determine the values of one or more of the following dental attribute(s): presence of tartar, presence of dental plaque, presence of a whitish tongue, lack of saliva. Each of these dental attributes is actually a bad breath index.

In step c), the processing computer selects one or more dental product(s) and/or dental care professional(s) relevant to the dental situation and additional information relating to the target consumer.

In particular, it can select the one or more relevant dental product(s) from a database of dental products, “DB_dental products”, as a function of the dental attribute values V determined in step b) and of the additional information Ic that it can retrieve from the “DB_Consumers” database by applying rules. If a dental product meets the set of rules, it is designated “relevant”.

The rules can be filters that determine whether a dental product is compatible with the one or more dental attribute value(s) determined in step b) and with the additional information. A rule can involve the dental situation of the target consumer, i.e., the value of at least one dental attribute. For example, “if the dental attribute value is the “presence of tartar”, selecting the following dental products: . . . ”, or “if the target consumer is wearing an orthodontic appliance, exclusively selecting a dental product from the following list: . . . ”, or “if the target consumer regularly experiences gingivitis, exclusively selecting a dental product from the following list: . . . ”.

A rule can involve a constraint included in the additional information. For example, “if a maximum price has been defined in the constraints, exclusively selecting dental products with a price below this maximum price”.

A rule can involve a dental objective. For example, “if the dental objective is to “improve breath”, exclusively selecting dental products from the following list: . . . ”.

A rule can involve a preference or requirement of the target consumer. For example, “if the target consumer requires a manual toothbrush, exclusively selecting a toothbrush from the following list: . . . ”, or “if the target consumer requires a 500 ml amount of a consumable, exclusively selecting a dental product from the following list: . . . ”.

A rule can involve a specific feature of the target consumer. For example, “if the target consumer is less than 15 years old, exclusively selecting dental products with less than XXX mg of fluorine”.

A rule can involve a relevant dental product already selected during the same step c). For example, “if the dental product XX is still considered to be potentially relevant, excluding the following dental products: . . . ”.

A rule can involve one or more events prior to step a), for example:
prior selection of a dental product: “if, following a previous request, dental product XXX has been selected, exclusively selecting a dental product from the following list: . . . ”;
the reaction of the target consumer following the response sent to them during a previous execution of the method according to the invention, for example, a purchase or a non-purchase of a relevant dental product proposed then: “if, following a previous execution of the method, dental product YY was selected and was not purchased, exclusively selecting a dental product from the following list: . . . ”, or “if the target consumer did not purchase the relevant dental products selected following a previous execution of the method, these dental products cannot be selected as a dental product”;
the frequency of implementing the method according to the invention by the target consumer, for example: “if the target consumer executes the method more than once a month, exclusively selecting dental products from the following list: . . . ”;
an evolution of the dental situation of the target consumer, for example: “if the value of dental attribute AA has positively evolved by more than x % per month, exclusively selecting dental products from the following list: . . . ”;

In one embodiment, the reaction can be assessed by analyzing the updated images. In particular, the non-purchase of the proposed relevant dental products in response to previous requests sometimes can be deduced from an analysis of an updated image or of successive updated images.

A rule can involve one or more data items independent of the target consumer or “external data”, for example, one or more reference data items, for example:
marketing data, for example, “if the target consumer is in social class XXX, exclusively selecting dental products from the following list: . . . ”;
statistical data, for example, derived from statistical processing of reactions of other target consumers to responses generated in accordance with the invention, for example: “if, on average, the other target consumers in the age range of the target consumer and with the same gender as the target consumer have not purchased dental product XX when it has been selected, excluding this dental product”.

The external data also can be data relating to the updated instant, for example: “if the updated instant ranges between the date D1 and the date D2, exclusively selecting the dental products sold with a promotion”.

A rule can involve several variables, for example, a dental objective and an event prior to step a), for example: “if the dental objective is “improving breath” and, following a previous execution of the method, dental product YY has been selected and purchased, exclusively selecting a dental product from the following list: . . . ”.

The above rules have a filtering function, i.e., they make dental products from the database of dental products irrelevant. The exclusive application of “filtering” rules can result in an empty set (no dental product is relevant) or in a set comprising several dental products, or even too many relevant dental products.

Preferably, the rules include one or more rule(s) with a weighting function. A “weighting” rule can assign a weight to one or more dental product(s) of the database of dental products, or can modify this weight, which allows the relevant dental products to be classified as a function of their relevance.

Instead of excluding a dental product such as a filtering rule, an equivalent weighting rule assigns a low weight thereto. Instead of limiting the selection to an exclusive list of dental products, a weighting rule assigns a high weight thereto. A weighting rule advantageously allows more precise assessment of the relevance of the dental products. A dental product is no longer 100% relevant or irrelevant, but becomes relevant to x %, with x ranging between 0 and 100%.

Examples of weighting rules are as follows:
“if the dental attribute value is the “presence of tartar”, assigning the weight P1 to the following dental products: ( . . . ) and the weight P2 to the following dental products: ( . . . )”;
“if a maximum price has been defined in the constraints, assigning a weight determined as a function of the difference between the price of a dental product and the maximum price”;
“if the dental objective is “improving breath”, assigning an increasing weight as a function of the mint flavor content”;
“if the target consumer prefers a manual toothbrush, assigning a weight of 90% to manual toothbrushes and a weight of 5% to electric toothbrushes”;
“if following a previous execution of the method, dental product XXX has been selected, assigning the following weights ( . . . ) to the following dental products ( . . . ), respectively”;
“if, following a previous execution of the method, dental product YY has been selected and has not been purchased, assigning a weight of x % to this dental product”;
“if the value of dental attribute AA has positively evolved by more than x % per month, assigning the following weights ( . . . ) to the following dental products ( . . . ), respectively”;
“if the target consumer is in social class XXX, assigning the following weights ( . . . ) to the following dental products ( . . . ), respectively”;
“if on average, the other target consumers in the age range of the target consumer and with the same gender as the target consumer have not purchased dental product XX when it has been selected, assigning the weight . . . thereto”;
“if the updated instant ranges between the date D1 and the date D2, assigning a weight . . . to the dental products sold with a promotion”.

Applying the rules allows one or more relevant dental product(s) to be selected from the database of dental products.

The database of dental products from which the relevant dental products are selected preferably comprises records for more than 100, more than 500 or more than 1,000, more than 5,000 or more than 10,000 dental products and/or less than 1,000,000 dental products.

Each record conventionally includes an identifier for a respective dental product and a set of values for the variables involved in the rules.

For example, a record for a dental product can include values for the following variables:
identifier,
brand;
manufacturer,
country of manufacture;
abrasive nature;
whitening nature;
amount of consumable, for example, for a toothpaste;
composition of the consumable, for example, for a toothpaste;
hardness, for example, for a toothbrush;
bristle density, for a toothbrush;
nature of the packaging;
price;
on promotion or not;
availability or delivery period;
list of other incompatible dental products;
features of the consumer purchasing the dental product the most;
affected markets or regions;
regulatory labels.

A record can also include information relating to the dental product and that is not included in the rules, for example, directions for use.

The use of rules is a “deterministic” approach, i.e., the rules always apply in the same way. Alternatively, the dental products of the database of dental products can be filtered and/or weighted by means of a neural network, called “selection neural network”. To this end, the selection neural network is trained by presenting it with sets, as input, comprising one or more dental attribute value(s) determined in a step b), and optionally with additional information, and, as output, with respective selections of dental products, preferably assigned a respective weight, for example, determined by means of the rules and/or by virtue of human expertise. The neural network thus learns to provide, for such a set of values determined for a step a), a selection of dental products.

In one embodiment, the selection neural network is trained by presenting it with sets, as input, each comprising one or more image(s) and optionally with additional information, and, as output, with respective selections of dental products, preferably assigned a respective weight, for example, determined by means of said rules. The selection neural network thus learns to provide a selection of dental products for one or more image(s). The selection neural network is preferably selected from among classification neural networks of the CNN or Fully Connected type.

Preferably, the dedicated application can be configured to define a maximum number of dental products in the selection. If the rules or the selection neural network result in a number of dental products that is greater than this maximum number, a final filter can be applied to retain only a portion of the dental products. The selection of the retained dental products can be arbitrary. Preferably, the rules or the selection neural network allow the dental products to be weighted as a function of their suitability for the dental situation of the target consumer and of the additional information. The final filter then retains the one or more dental product(s) that have been assigned the highest weights.

As an alternative to a selection neural network, a simple decision algorithm can be implemented.

The selection of a dental care professional from a database of dental care professionals can be carried out in a manner similar to the selection of a dental product from the database of dental products.

A record for a dental care professional can include, for example, values for the following variables:
identifier, for example, name and forename;
specialty/diploma(s);
equipment;
address;
spoken language(s);
opening times;
gender;
age;
the identification of one or more product(s) that the dental care professional can provide;
an assessment score, for example, by the target consumers who have consulted the dental care professional.

Rules or a selection neural network can be used to select a dental care professional as a function of the value of the dental attribute and/or of additional information, as described above for the selection of a dental product.

In step d), the target consumer can be presented with a response to advise them of the one or more selected dental product(s) and, preferably, to provide them with directions for their use.

If the processing computer is not integrated in the mobile telephone, the response is sent to the mobile telephone by the processing computer, preferably by means of a mobile telephone network. The response is preferably a message, for example, an SMS or an e-mail.

The response is then shown on the mobile telephone. Advantageously, the target consumer therefore quickly receives a response to their request that is well suited to their dental situation.

Preferably, if a plurality of dental products has been selected and they are assigned a respective weight, they are preferably shown as a function of their relevance, for example, by showing the most relevant dental products at the beginning of the response.

Preferably, the response includes advice, preferably determined as a function of the one or more relevant dental product(s), and preferably as a function of the value of the dental attribute. The advice can be derived from records in the database of dental products.

Preferably, it includes information concerning the use of the relevant dental products and/or of the method, for example, by recommending a date for the next execution thereof.

The response can also contain information relating to one or more relevant dental care professional(s).

Preferably, in particular when the target consumer has entered that they wish to achieve a dental objective, the response can notify them of the date scheduled for the next execution of the method and/or a percentage of the progress of the method in order to reach the dental objective.

In one embodiment, a package containing the one or more selected dental product(s) is prepared and sent to the target consumer. Preferably, a message is sent to the mobile telephone to notify them that the package has been sent.

In one embodiment, the target consumer has taken out a subscription and, at a regular interval, is asked to implement a method according to the invention. They thus regularly receive a package, for example, with a new toothbrush and a new toothpaste better suited to their dental situation at the acquisition instant.

In one embodiment, as a function of the dental situation as assessed in step b), the dedicated application can automatically contact a dental care professional, for example, for a videoconferencing consultation, or can recommend a dental care professional, preferably selected as a function of the dental situation as assessed in step b) and/or of the geographical proximity to the target consumer, preferably a relevant dental care professional.

As is now clearly apparent, the method of the invention allows a target consumer to automatically obtain advice for purchasing a dental product or for selecting a dental care professional. This advice is advantageously personalized since it depends on the updated image and on additional information allowing the target consumer to specify their need. Without being a specialist in dental products, the target consumer is therefore capable of selecting dental products well suited to their situation.

Of course, the invention is not limited to the embodiments described and represented above.

In one embodiment, the database of dental products only includes records for dental products that are exclusively intended to improve the dental situation.

In another embodiment, the database of dental products further comprises records for dental products that are not exclusively intended to improve the dental situation, i.e., without a therapeutic or prophylactic effect. For example, it can contain records relating to foods that are beneficial to consume under certain circumstances, for example, as a function of the value of a dental attribute and/or as a function of another selected dental product and/or as a function of a dental objective to be achieved.

For example, if an “improving breath” dental objective has been set, particular foods can be recommended. For example, a rule can establish that, “if the “improving breath” dental objective has been set, the following dental products ( . . . ), including foods ( . . . ), are to be selected”. A record relating to a food can contain directions for preparing this food. Moreover, an acquisition device other than a mobile telephone can be used for step a).

In one embodiment, the acquisition device, and preferably the processing computer, are fixed relative to the ground, preferably in the store, for example, the supermarket, in which the target consumer is located, preferably in the vicinity of the dental products.

The image acquisition appliance, for example, a camera or an imaging device, can be arranged behind a one-way mirror, which may or may not be fixed relative to the ground. Preferably, a screen, preferably integrated in the mirror, provides instructions for guiding the target consumer when acquiring the images. Preferably, a detector detects the presence of the target consumer in front of the mirror and launches the dedicated application accordingly.

In particular, such a mirror can be arranged in an aisle containing dental products. The target consumer positions themselves in front of the mirror and is detected by the detector. An “acquisition computer”, preferably the processing computer, then guides the target consumer so that they are positioned in a predetermined position, for example, facing the image acquisition appliance, with the head facing the left, and/or so as to sufficiently find their lips. Preferably, the acquisition computer determines, for example, by means of a neural network, whether the position of the target consumer is suitable for acquiring an image and, in this case, preferably, triggers the acquisition of an updated image.

In one embodiment, the screen of the mirror allows additional information to be acquired. In a preferred embodiment, a facial recognition algorithm implemented by the acquisition computer identifies the target consumer without the consumer having to interact with the mirror. The identifier, which is sent to the processing computer, then allows said processing computer to access the “DB_Consumers” database in order to supplement it and/or to extract additional information related to the target consumer.

Facial recognition advantageously allows, in step d), a relevant dental product to be delivered and/or a response to be provided and/or a relevant dental care professional to be recommended, by taking into account prior uses of the acquisition device by the target consumer and/or additional information originating from other additional information sources, without the target consumer having contact with the acquisition device. They simply need to position themselves in front of the mirror. Hygiene conditions and ergonomics are improved.

For example, if the “DB_Consumers” database contains the additional information by which the target consumer has recently purchased an electric toothbrush, the acquisition device can advise them, as soon as they look into the mirror, to purchase a toothpaste specific to this toothbrush, preferably by informing them that this toothpaste is particularly suitable for said toothbrush.

The acquisition computer can then act as a processing computer, and can show, preferably on the screen of the mirror, the response, in particular for recommending a dental product. In a few seconds, simply by smiling in front of the mirror, the target consumer can thus receive a recommendation for selecting the dental product, in particular a toothpaste or a toothbrush.

The processing computer also can be remote from the acquisition computer. This computer then prepares and transmits, to the remote processing computer, a request containing the updated image and optional additional information. After processing the request, the processing computer returns a response to the acquisition computer to be shown to the target consumer, preferably on the screen of the mirror.

A method according to the invention can be used for many different products.

According to various embodiments:
the dental objective is to whiten the teeth, the dental organ is a tooth, the dental attribute is a shade, for example, a whiteness, of the dental organ, and the relevant dental product is a tooth whitening product and/or a specific toothpaste and/or a specific tooth brush; or
the dental objective is to reduce dental plaque, the dental organ is a tooth, the dental attribute is an amount and/or a size and/or a color of dental plaque, and the relevant dental product is an anti-tartar product, and/or a toothpaste and/or a toothbrush; or
the dental objective is to reduce soft tissue inflammation, the dental organ is the gum, the dental attribute is a color and/or a size of an inflamed area, and the relevant dental product is an anti-gingivitis product; or
the dental objective is to improve breath, the dental organ is a tooth, the dental attribute is the presence of tartar, and/or the presence of dental plaque, and/or the presence of food debris, and the relevant dental product is an anti-tartar product and/or an oral lotion for preventing bad breath, and/or a toothpaste and/or a toothbrush; or
the dental objective is to improve breath, the dental organ is the tongue, the dental attribute is a color of the dental organ, and the relevant dental product is an anti-tartar product or an oral lotion for preventing bad breath, and/or a toothpaste and/or a toothbrush; or
the dental objective is to improve breath, the dental organ is the mouth, the dental attribute is an amount of saliva in the mouth, and the relevant dental product is an anti-tartar product or an oral lotion for preventing bad breath, and/or a toothpaste and/or a toothbrush; or
the dental objective is to reduce whitish deposits, the dental organ is the mouth, the dental attribute is an amount of saliva, and the relevant dental product is an oral lotion, in particular an artificial saliva; or
the dental objective is to improve the general appearance of the smile, the dental organ is a lip or the lips, the dental attribute is a shape and/or a thickness and/or a color of the lip, and the relevant dental product is a product for restructuring the lips; or
the dental objective is to improve the general appearance of the smile, the dental organ is the perioral area, the dental attribute is a shape and/or a thickness and/or a color of bitterness folds, and/or a thickness and/or a color of nasogenian furrows, and/or a thickness and/or a color of sun wrinkles, and the relevant dental product is a wrinkle reduction product.

In an alternative embodiment of the invention, a method according to the invention can be used to select an orthodontic treatment or a diet or an orthodontic appliance or a functional apparatus, rather than a dental product.

According to various embodiments of this alternative embodiment:
the dental objective is to improve breath, the dental organ is a tooth, the dental attribute is the presence of tartar, and/or the presence of dental plaque, and/or the presence of food debris, and, in step c), instead of or in addition to the relevant dental product, a relevant diet is selected, and then, in step d), said relevant diet is presented; or
the dental objective is to correct/improve the alignment of the teeth, the dental organ is one or two arches, the dental attribute is a congestion index and/or an occlusion class, and/or a vertical overhang value, and/or a horizontal overhang value, and, in step c), instead of or in addition to the relevant dental product, a relevant type of orthodontic treatment and/or a duration of a relevant orthodontic treatment is selected, and then, in step d) said relevant type of orthodontic treatment and/or said relevant orthodontic treatment duration are presented; or
the dental objective is to reduce snoring, the dental organ is the tongue, the dental attribute is a position of the tongue, and, in step c), instead of or in addition to the relevant dental product, a relevant functional apparatus is selected, and then, in step d), said relevant functional apparatus is presented; or
the relevant dental product is a functional apparatus; or
the dental objective is to reduce sleep apnea, the dental organ is the tongue, the dental attribute is a position of the tongue, and, in step c), instead of or in addition to the relevant dental product, a relevant functional apparatus is selected, and then, in step d), said relevant functional apparatus is presented.

### Example of the Application of the Invention

A target consumer acquires an updated image with a mobile telephone. The updated image is analyzed by a processing computer, which can be the mobile telephone. The analysis allows at least one dental attribute value to be determined, preferably, the analysis allows a plurality of values to be determined for a dental attribute, preferably the analysis allows a plurality of values to be determined for a plurality of dental attributes.

For example, the analysis can determine one or more of the following value(s):
a shade value for one or more teeth depicted on the updated image;
a value for an amount and/or a color of dental plaque on one or more teeth depicted on the updated image;
a color and/or size value for one or more gums depicted on the updated image;
a value of the presence of tartar and/or food debris on one or more teeth depicted on the updated image;
a value for an amount of saliva inside the mouth depicted on the updated image;
a color value of the tongue depicted on the updated image;
a shape and/or thickness and/or color value of one or both lips depicted on the updated image;
a shape and/or appearance and/or thickness and/or color value of a perioral area depicted on the updated image;
a positioning value of the tongue depicted on the updated image.

The values can be quantitative or qualitative.

A value can be:
a Boolean;
a vector,
a figure or a number,
selected from among a set of predefined values, for example, determined on completion of a classification.

The values can be determined by conventional image processing algorithms. For example, by segmentation, classification methods, by using neural networks, by analyzing colors, contours, textures, by detection.

The analysis can particularly comprise:
segmenting the analysis image in order to distinguish various analysis areas depicted on the analysis image, the analysis areas can be a tooth, a gum, a tongue, the inside of the mouth, a lip, a perioral area; then,
for one or more analysis areas, determining a value for one or more dental attribute(s), for example, by means of neural networks:
   for a tooth, a value for the shade, the shape, the amount of dental plaque, the dental plaque size, the dental plaque color, the presence of tartar and/or the presence of food debris can be values of determined dental attributes;
   for a gum, a color, shape, and/or size value can be values of determined dental attributes;
   for a tongue, a color, in particular for the presence of whitish stains, positioning, and/or shape value can be determined dental attribute values;
   for the inside of the mouth, a value of the presence of saliva, of the amount of saliva and/or of the presence of food debris can be values of determined dental attributes;
   for a lip, a color, shape and/or thickness value can be values of determined dental attributes;
   for a perioral area, a value of the shape of bitterness folds, of the thickness of bitterness folds, of the color of bitterness folds, of the thickness of nasogenian furrows, of the color of nasogenian furrows, of the thickness of sun wrinkles and/or of the color of sun wrinkles can be values of determined dental attributes.

The target consumer can also enter additional information for facilitating the selection.

Preferably, the target consumer enters at least one dental objective as additional information.

In particular, the database is configured so that it lists, for each dental product contained in the database, the dental attribute values for which said dental product is relevant and/or the additional information for which said dental product is relevant.

Thus, as a function of the values determined when analyzing the updated image, and of any additional information, the processing computer can select one or more relevant dental product(s) with respect to the dental situation of the target consumer.

For example, determining a value for a dental attribute corresponding to the shade of at least one tooth can allow a tooth whitening product to be selected. Preferably, at least one dental objective is determined, preferably the at least one dental objective is to whiten the teeth.

In particular, based on the analysis of an updated image, a value relating to the shade of a tooth can be determined. If the shade is yellowish, for example, the processing computer can select at least one tooth whitening product from the database. Alternatively or additionally, a relevant dental product can be a toothpaste and/or a toothbrush.

Another example can be determining a value for a dental attribute corresponding to an amount of dental plaque on a tooth and/or a value for a dental attribute corresponding to a size of dental plaque on a tooth and/or a value for a dental attribute corresponding to a color of dental plaque on a tooth. If the amount of dental plaque on a tooth or a group of teeth depicted on the updated image exceeds a threshold value, with the threshold value being predefined in the database, then an anti-tartar product, and/or a toothpaste and/or a toothbrush can be selected as a relevant dental product(s). Preferably, at least one dental objective is determined, preferably the at least one dental objective is to reduce dental plaque.

Another example can be determining a value for a dental attribute corresponding to a color of the tongue, in particular the dental attribute value can correspond to the presence of whitish deposits on the tongue. If the analysis of the updated image determines the presence of whitish deposits on the tongue then a relevant dental product can be an anti-tartar product or an oral lotion for preventing bad breath. Preferably, at least one dental objective is determined, preferably the at least one dental objective is to improve breath. Additional information entered by the target consumer can be the consumption of drugs, with the entered drugs resulting in the reduction of the amount of saliva, or a state of dehydration.

Another example can be determining a value for a dental attribute corresponding to the positioning of the tongue, a functional apparatus for sleep apnea or for positioning the tongue can be selected, in addition to or instead of a relevant dental product. Preferably, at least one dental objective is determined, preferably the at least one dental objective is to reduce snoring. Alternatively, the at least one dental objective is to reduce sleep apnea. Determining a plurality of values for one or more attribute(s) of teeth can allow a plurality of relevant dental products to be selected. Alternatively, a single relevant dental product can be selected.

The one or more selected dental product(s) is/are presented to the target consumer, for example, displayed on the screen of their mobile telephone.

After displaying the one or more selected dental product(s), the consumer can advantageously decide to have one or more of the one or more relevant dental product(s) delivered.

1. A method for selecting at least one dental product for a target consumer, said method comprising the following steps:
a) acquiring, at an acquisition instant and with an acquisition device:
at least one image, called “updated image”, at least partially depicting a dental arch of the target consumer; and
b) analyzing the updated image by a processing computer, so as to determine a value for at least one dental attribute relating to the dental situation of the target consumer at the acquisition instant;
c) selecting, by the processing computer, as a function of said value,
at least one dental product, called “relevant dental product”, from a database of dental products;
d) presenting the target consumer with a response relating to said at least one relevant dental product; and/or
delivering said at least one relevant dental product to the target consumer;
in step b), the dental attribute being:
an appearance parameter for a gum, a tongue, one or more lips, a perioral area, a tooth or a group of teeth depicted on the updated image;
a parameter relating to the state of a gum, a tongue, one or both lips, a perioral area, a tooth or a group of teeth or the mouth depicted on the updated image;
a parameter relating to the shape of a gum, the tongue, one or both lips, a perioral area, a tooth or a group of teeth depicted on the updated image;
said at least one relevant dental product being selected from among toothpastes, toothbrushes, dental floss, chewing gums, interdental brushes, whitening kits, gum care products, products for preventing bad breath and oral lotions, lip restructuring or wrinkle reduction products.

2. The method as claimed in claim 1, wherein step a), is at least partially carried out by means of a dedicated application loaded into the acquisition device,
the acquisition device being:
a mobile telephone; or
a device comprising a mirror, a camera or an imaging device, and an acquisition computer.

3. The method as claimed in the claim 2,
comprising, before step a), a step in which the dedicated application is downloaded into the mobile telephone, with the downloading being triggered by reading a code printed on a dental product and/or on a panel arranged in an aisle in which dental products are arranged; or
comprising, in step a), the detection of the target consumer in front of the mirror and the acquisition of the updated image when the target consumer is detected in front of the mirror.

4. The method as claimed in claim 3, wherein, in step a), the dedicated application analyzes the content of a preview image and guides the positioning of the target consumer as a function of the result of said analysis.

5. The method as claimed in claim 17, wherein the first and/or second additional information comprises additional information:
defining a constraint or a preference to be imposed for the selection in step c); and/or
defining a constraint or a preference to be imposed for the presentation and/or the delivery and/or the placing in contact in step c); and/or
defining a dental objective to be achieved by the target consumer; and/or
relating to the target consumer.

6. The method as claimed in claim 5, wherein the additional information defining a constraint specifies:
a maximum price, with the price of a dental product having to be below said maximum price so that said dental product can be selected in step c);
when the dental product comprises a consumable, a range for the amount of the consumable, within which range the amount of consumable of a dental product must be included so that said dental product can be selected in step c);
a maximum period for delivering or providing a dental product so that said dental product can be selected in step c);
a list of one or more ingredient(s) that a dental product must not include or must include so that said dental product can be selected in step c);
a list of one or more technical feature(s) that a dental product must not include or must include so that said dental product can be selected in step c);
a list of manufacturing countries where a dental product must be manufactured or must not be manufactured so that said dental product can be selected in step c);
a list of manufacturers by which a dental product must be manufactured or must not be manufactured so that said dental product can be selected in step c);
a list of brands that must include the brand of a dental product so that said dental product can be selected in step c);
a maximum price, with the price for a service by a dental care professional having to be below said maximum price so that said dental care professional can be selected in step c);
a maximum period for making an appointment with the dental care professional so that said dental care professional can be selected in step c);
a list of one or more item(s) of equipment that a dental care professional must have available so that said dental care professional can be selected in step c);
a list of one or more diploma(s) or qualification(s) that a dental care professional must hold so that said dental care professional can be selected in step c);
a list of language(s) in which a dental care professional must be proficient so that said dental care professional can be selected in step c);
criteria for presenting the response in step d);
criteria for delivering the one or more relevant dental product(s) in step d);
criteria for contacting the relevant dental care professional in step d); and/or
the dental objective is selected from among the following objectives: teeth whitening, reducing plaque, reducing tartar, reducing a soft tissue inflammation, improving breath, correcting/improving teeth alignment, improving the general appearance of the smile, reducing snoring and/or sleep apnea, and/or improving lip shape and/or improving the appearance of the perioral area; and/or
the additional information relating to the target consumer specifies:
their age; and/or
their gender; and/or
the one or more dental care professional(s) they have already consulted or they consult regularly; and/or
whether or not they are wearing an orthodontic appliance; and/or
if they are predisposed to certain pathologies; and/or
if they have followed or if they follow or if they are contemplating following a particular orthodontic treatment; and/or
if they are currently undergoing dental treatment in order to achieve a dental objective and/or, if they have already previously used the method within the context of said dental treatment, details concerning said dental treatment; and/or
whether or not they are content with their smile, their breath or their current dental situation; and/or
if they experience pain, and/or a measure of the perceived pain, and/or a location of the perceived pain; and/or
if their teeth are sensitive to contact with heat and/or cold.

7. The method as claimed in claim 17, wherein, in step a), the acquired first additional information is stored in said database, and/or wherein second additional information is provided by one or more different additional information source(s) of the acquisition device.

8. The method as claimed in claim 1, wherein, in step b), the following steps are carried out:
segmenting by means of a “segmentation” neural network, so as to isolate the depiction of at least one dental organ on the updated image;
determining the value of the dental attribute by submitting said depiction as input for a “valorization” neural network that is trained to provide, as output, a value of said dental attribute in response to the presentation, as input, of a representation of one of said dental organs.

9. The method according to claim 8, wherein:
the dental objective is to whiten the teeth, the dental organ is a tooth, the dental attribute is a shade, for example, a whiteness, of the dental organ, and the relevant dental product is a tooth whitening product; or
the dental objective is to reduce dental plaque, the dental organ is a tooth, the dental attribute is an amount and/or a size and/or a color of dental plaque, and the relevant dental product is an anti-tartar product, and/or a toothpaste and/or a toothbrush; or
the dental objective is to reduce soft tissue inflammation, the dental organ is the gum, the dental attribute is a color and/or a size of an inflamed area, and the relevant dental product is an anti-gingivitis product; or
the dental objective is to improve breath, the dental organ is a tooth, the dental attribute is the presence of tartar, and/or the presence of dental plaque, and/or the presence of food debris, and the relevant dental product is an anti-tartar product and/or an oral lotion for preventing bad breath, and/or a toothpaste and/or a toothbrush; or
the dental objective is to improve breath, the dental organ is the tongue, the dental attribute is a color of the dental organ, and the relevant dental product is an anti-tartar product or an oral lotion for preventing bad breath, and/or a toothpaste and/or a toothbrush; or
the dental objective is to improve breath, the dental organ is the mouth, the dental attribute is an amount of saliva in the mouth, and the relevant dental product is an anti-tartar product or an oral lotion for preventing bad breath, and/or a toothpaste and/or a toothbrush; or
the dental objective is to reduce whitish deposits, the dental organ is the mouth, the dental attribute is an amount of saliva, and the relevant dental product is an artificial saliva; or
the dental objective is to improve the general appearance of the smile, the dental organ is a lip or the lips, the dental attribute is a shape and/or a thickness and/or a color of the lip, and the relevant dental product is a product for restructuring the lips; or
the dental objective is to improve the general appearance of the smile, the dental organ is the perioral area, the dental attribute is a shape and/or a thickness and/or a color of bitterness folds, and/or a thickness and/or a color of nasogenian furrows, and/or a thickness and/or a color of sun wrinkles, and the relevant dental product is a wrinkle reduction product.

10. The method as claimed in claim 1, wherein, in step c), a set of rules is applied to the value determined in step b), with said set of rules comprising at least one rule selected from among:
filtering rules suitable for retaining dental products from the database of dental products and/or for retaining dental care professionals from the database of dental care professionals, as a function of the value determined in step b); and
weighting rules suitable for giving weights to the dental products of the database of dental products and/or to the dental care professionals of the database of dental care professionals, as a function of the value determined in step b).

11. The method as claimed in claim 10, wherein the rules involve,
said value determined in step b); and/or
a constraint and/or a dental objective and/or a preference and/or a requirement of the target consumer and/or a specific feature of the target consumer and/or a feature of an event prior to step a), and/or a plurality of dental products and/or a plurality of dental care professionals and/or data independent of the target consumer.

12. The method as claimed in claim 1, wherein, in step c), a selection neural network is implemented in order to provide, as output, one or more relevant dental product(s) as a function of the value determined in step b) introduced at the input of said selection neural network.

13. The method as claimed in claim 1, wherein, at step c), in addition to the at least one dental product, at least one dental care professional, called “relevant dental care professional”, is selected from a database of dental care professionals and in step d), the target consumer and a dental care professional are placed in contact as a function of the value determined in step c).

14. The method as claimed in claim 1, wherein the database of dental products includes records for dental products that are not exclusively intended to improve a dental situation.

15. The method as claimed in claim 1, wherein the acquisition in step a) and/or the presentation in step d), is/are carried out at the home of the target consumer or in a store or a pharmacy in which said dental products are offered for sale or in which teeth whitening can be carried out.

16. The method as claimed in claim 1, wherein step a), comprises the acquisition of first additional information; and step c) the selection is performed as a function of said value, and of said first additional information.

17. The method as claimed in claim 16, wherein at step c) the selection is further performed as a function of second additional information that is stored, prior to step a), in a database.

18. The method as claimed in claim 1, wherein at step c), in addition to the at least one dental product, at least one dental care professional, called “relevant dental care professional”, is selected from a database of dental care professionals and step d) comprises the presentation of the target consumer with a response relating to said at least one relevant dental product and to said at least one relevant dental care professional; and/or
delivering said at least one relevant dental product to the target consumer; and/or
placing the target consumer in contact with a dental care professional selected in step c).
